(19) United States
(12) Patent Application Publication
(10) Pub. No.: US 2025/0120877 A1
(43) Pub. Date: Apr. 17, 2025

(54) WEARABLE DEVICE TO TREAT TREMOR

(71) Applicant: Equibands Inc., Vernon Hills, IL (US)

(72) Inventor: Maanav NARULA, Highland Park, IL (US)

(21) Appl. No.: 18/929,252

(22) Filed: Oct. 28, 2024

Related U.S. Application Data
(62) Division of application No. 17/492,062, filed on Oct. 1, 2021.
Application No. 17/492,062 is a continuation of application No. PCT/US20/26393, filed on Apr. 2, 2020.
(60) Provisional application No. 62/829,783, filed on Apr. 5, 2019.

(51) Int. Cl. A61H 23/00; A61F 5/01

(52) U.S. Cl. CPC A61H 23/004; A61F 5/0118; A61H 2201/0165; A61H 2201/1207; A61H 2201/1436; A61H 2201/149; A61H 2201/1635; A61H 2201/165; A61H 2205/065

(57) ABSTRACT

Devices, systems, and methods are provided to treat tremor in an outer extremity, typically a hand, of a subject. A wearable base or glove is provided with one or more tremor damping mechanisms, which can be of different or the same types, in the case of a plurality of tremor damping mechanisms. One or more frictional damping mechanisms can be provided and/or one or more tuned mass damping mechanisms can be provided. The frictional dampening mechanism can simply be the viscoelastic material of the wearable base that deforms and interferes with tremor movement. The frictional dampening mechanism can be one or more tension elements provided within the body of the wearable base. The tuned damping mechanism may comprise one or more resonators held within a housing coupled to the wearable base. The tremor damping mechanisms can be self-adjusting and/or adjustable by the wearer.

### CROSS-REFERENCE

This application is a divisional application of U.S. application Ser. No. 17/492,062, filed Oct. 1, 2021, which is a continuation of PCT Application No. PCT/US20/26393, filed Apr. 2, 2020, which claims the benefit of U.S. Provisional Application No. 62/829,783, filed Apr. 5, 2019, each of which is incorporated herein by reference in its entirety.

### BACKGROUND

The present disclosure relates to medical devices, systems, and methods, particularly for treating tremor in outer extremities of patients, such as hand tremors.

Hand tremors are common symptoms of neurological disorders such as Parkinson's Disease and Essential Tremor. One common tremor motion is a rotation or pivoting of the hand up and down about the wrist and another common tremor motion is a rotation or pivoting of the hand about the “rolling axis”, an axis that passes through the middle of the wrist and the middle finger. Worldwide, over 80 million people are affected by hand tremors. Such tremors can adversely affect the quality of life of many patients, making daily activities such as brushing teeth, eating, cleaning, writing, handling objects, to name a few, more difficult and inconvenient. Drug therapies to treat tremors can be expensive and result in numerous adverse side effects. Electro-mechanical and mechanical devices to treat tremor are also available, but many are bulky, intrusive, heavy, uncomfortable, difficult to adjust, and/or otherwise unsatisfactory. Hence, there are needs for improved devices, systems, and methods to treat hand tremor.

Patents and published patent applications that are relevant include, but are not limited to: U.S. Pat. Nos. 5,058,571, 6,458,089, 6,695,794, 6,730,049, US2018266820, and US2019059733.

### SUMMARY

Systems, devices, and methods to treat tremor in outer extremities of patients are disclosed herein. In particular, disclosed is a wearable device that counteracts and reduces the amplitude of hand tremors, using one or more damping mechanisms including tuned mass dampers and frictional damping. The wearable device may be configured to be worn on the distal forearm, hand, and/or wrist of a patient. The wearable device may include a frictional damping mechanism and may be coupled to one or more tuned mass dampers. The amount of vibrational damping provided by these mechanisms can be adjusted by the patient or other user. The wearable device may also calibrate itself, for instance when charged or otherwise powered, to account for tremor variation during and across tremor episodes.

Aspects of the present disclosure provide apparatuses to treat tremor in an outer extremity of a subject. An exemplary apparatus may be provided with one or more tremor damping mechanisms of different types. The apparatus may comprise a wearable base, a frictional damping mechanism, a tuned mass damping mechanism, a housing, and a plurality of resonators held within the housing. The wearable base may be configured to be worn over at least a joint of the outer extremity. The wearable base may have a proximal fixed region and a distal moving region. The frictional damping mechanism may be coupled to the wearable base and be configured to damp movement of the distal moving region relative to the proximal fixed region in response to tremor movement in the outer extremity. The tuned mass damping mechanism may be coupled to the wearable base. The tuned mass damping mechanism may comprise a housing coupled to the wearable base and a plurality of resonators, typically held within the housing. The plurality of resonators may be configured to destructively interfere with the tremor movement in the outer extremity, such as by being movable within the housing. In some cases, the housing may act as an outer resonator itself. The outer extremity will typically be a hand of the subject. The wearable base may be configured to be worn over a wrist and at least a portion of the hand of the patient, and sometimes a distal forearm of the patient.

The frictional damping mechanism may comprise a viscoelastic material of the wearable base. The viscoelastic material may be configured to deform and interfere with the tremor movement in response to the tremor movement. The frictional damping mechanism may further comprise a flexoelectric material of the wearable base. The flexoelectric material may also be configured to deform and interfere with the tremor movement in response to the tremor movement. Alternatively or in combination, the frictional damping mechanism may comprises at least one tension element within a body of the wearable base. In response to the tremor movement, the at least one tension element may apply a force opposite in direction to the tremor movement to damp the movement of the distal moving region relative to the proximal fixed region. The at least one tension element may comprise at least one belt, wire, or rope. The at least one tension element may comprise a plurality of tension elements. The ends of the at least one tension element may be fixedly attached to the distal moving region of the wearable base. The frictional damping mechanism may further comprise at least one capstan at the proximal fixed region coupled to the at least one tension element. The at least one tension element may be wrapped around the at least one capstan. The frictional damping mechanism may further comprise at least one adjustment element coupled to the at least one capstan to increase or decrease an amount of tension the at least one tension element is held in within the wearable base.

The plurality of resonators may comprise a first resonating mass and a first spring element coupling the first resonating mass to the housing. The plurality of resonators may further comprise an adjustment element to adjust a spring constant of the first spring element. The adjustment element may comprise one or more of a motor or an actuator coupled to the first spring element and may be configured to selectively tighten or restrict movement of the first spring element. Another adjustment and/or calibration element may be provided by a mechanism that controls the number of springs acting on the resonators. Another adjustment and/or calibration element may be provided by a variable fluid damper mechanism, for example. Another adjustment element and/or calibration element may be provided by antagonistic controlled stiffness spring systems. The plurality of resonators may comprise a second resonating mass and a second spring element. The second resonating mass and a second spring element may be held and movable within the housing of the tuned mass damping mechanism. The second resonating mass and a second spring element may be held and movable within the first resonating mass. At least two resonators of the plurality of resonators may be embedded, arranged in parallel, or arranged in series with respect to one another. Noise damping material may be provided within the tuned mass damping mechanism.

The tuned mass damping mechanism may be detachable coupled to the wearable base. The wearable base may be configured to detachably couple to a plurality of tuned mass damping mechanisms. The wearable base may be configured to detachably couple to a first tuned mass damping mechanism at a first side of the wearable base and a second tuned mass damping mechanism at a second side of the wearable base. The tuned mass damping mechanism is detachably coupled to the wearable base with a rotational to linear motion mechanism, such as a slider-crank mechanism and/or a Scotch yoke mechanism. One or more torsional pendulums may act as additional resonators to increase tremor damping. One or more slider pieces may be used as intermediaries between the hand and resonator(s) to transmit the force of the tremor to the tuned mass damper mechanisms.

Another exemplary apparatus to treat tremor in an outer extremity of a subject may comprise a wearable base, a tuned mass damping mechanism only, with a housing and a plurality of resonators held, typically held within the housing. The wearable base may be configured to be worn over at least a joint of the outer extremity. The tuned mass damping mechanism may be coupled to the wearable base. The housing may be coupled to the wearable base. The plurality of resonators may be configured to destructively interfere with the tremor movement in the outer extremity, such as by being moveable within the housing. In some cases, the housing may act as an outer resonator itself. The plurality of resonators may comprise a first resonating mass and a first spring element coupling the first resonating mass to the housing. The plurality of resonators may further comprise an adjustment element to adjust a spring constant of the first spring element. The outer extremity will typically be a hand of the subject. The wearable base may be configured to be worn over a wrist and at least a portion of the hand of the patient, and sometimes a distal forearm of the patient.

The adjustment element may comprise an actuator or a motor coupled to the first spring element and configured to selectively tighten or restrict movement of the first spring element.

The plurality of resonators may comprise a second resonating mass and a second spring element. The second resonating mass and a second spring element may be held and movable within the housing of the tuned mass damping mechanism. The second resonating mass and a second spring element may be held and movable within the first resonating mass. At least two resonators of the plurality of resonators may be embedded, arranged in parallel, or arranged in series with respect to one another. Noise damping material may be provided within the tuned mass damping mechanism.

The tuned mass damping mechanism may be detachably coupled to the wearable base. The wearable base may be configured to detachably couple to a plurality of tuned mass damping mechanisms. The wearable base may be configured to detachably couple to a first tuned mass damping mechanism at a first side of the wearable base and a second tuned mass damping mechanism at a second side of the wearable base. A variety of attachment points are contemplated, including the top, bottom, left, and right of the wearable base. The tuned mass damping mechanism is detachably coupled to the wearable base with a rotational to linear motion mechanism, such as a slider-crank mechanism and/or a Scotch yoke mechanism. The tuned mass damper mechanism may include one or more torsional pendulums and/or one or more slider pieces as an intermediary between the hand and resonator(s).

Another exemplary apparatus to treat tremor in an outer extremity of a subject may comprise a wearable base and a frictional damping mechanism only. The wearable base may be configured to be worn over at least a joint of the outer extremity. The wearable base may have a proximal fixed region and a distal moving region. The frictional damping mechanism may be coupled to the wearable base and be configured to damp movement of the distal moving region relative to the proximal fixed region in response to tremor movement in the outer extremity. The frictional damping mechanism may comprise at least one tension element held in tension within a body of the wearable base. In response to the tremor movement, the at least one tension element may apply a force opposite in direction to the tremor movement to damp the movement of the distal moving region relative to the proximal fixed region. The outer extremity will typically be a hand of the subject. The wearable base may be configured to be worn over a wrist and at least a portion of the hand of the patient, and sometimes a distal forearm of the patient.

The frictional damping mechanism may further comprise a viscoelastic material of the wearable base. The viscoelastic material may be configured to deform and interfere with the tremor movement in response to the tremor movement.

The at least one tension element may comprise at least one belt, wire, or rope. The at least one tension element may comprise a plurality of tension elements. The ends of the at least one tension element may be fixedly attached to the distal moving region of the wearable base. The frictional damping mechanism may further comprise at least one capstan at the proximal fixed region coupled to the at least one tension element. The at least one tension element may be wrapped around the at least one capstan. The frictional damping mechanism may further comprise at least one adjustment element coupled to the at least one capstan to increase or decrease an amount of tension of tension the at least one tension element is held in within the wearable base.

Another exemplary apparatus to treat tremor in an outer extremity of a subject may comprise a wearable base and a frictional damping mechanism. The wearable base may be configured to be worn over at least a joint of the outer extremity. The wearable base may have a proximal fixed region and a distal moving region. The frictional damping mechanism may be configured to damp movement of the distal moving region relative to the proximal fixed region in response to tremor movement in the outer extremity. The frictional damping mechanism may comprise a viscoelastic material of the wearable base. The viscoelastic material may be configured to deform and interfere with the tremor movement in response to the tremor movement. The outer extremity is a hand of the subject. The wearable base may be configured to be worn over a wrist and at least a portion of the hand of the patient, and sometimes a distal forearm of the patient.

Another aspect of the present disclosure provides methods of treating tremor in an outer extremity of a subject. In an exemplary method, a wearable base to be worn over at least a joint of the outer extremity may be provided, movement of a distal moving region of the wearable base worn on the outer extremity relative to a proximal fixed region of the wearable base may be damped in response to tremor movement in the outer extremity using a frictional damping mechanism, and movement of the outer extremity may be damped using a tuned mass damping mechanism coupled to the wearable base worn on the outer extremity. The wearable base may be worn over a wrist and at least a portion of a hand of the subject, and sometimes a distal forearm of the patient.

Movement may be dampened using the frictional damping mechanism by applying a force opposite in direction to the tremor movement in response to the tremor movement with the frictional damping mechanism. The force opposite in direction to the tremor movement may be applied by a viscoelastic material of the wearable base. The viscoelastic material may be configured to deform and interfere with the tremor movement in response to the tremor movement. Alternatively or in combination, the force opposite in direction to the tremor movement may be applied by at least one tension element held in tension within a body of the wearable base. The amount of tension of the at least one tension element may be adjusted.

Movement may be dampened using the tuned mass damping mechanism by providing a plurality of resonators held within a housing coupled to the wearable base. Movement may be dampened using the tuned mass damping mechanism by oscillating a plurality of resonating masses within the plurality of resonators. The amount of oscillation allowed to at least one resonator of the plurality of resonators may be adjusted. The plurality of resonators may comprise a first resonating mass and a second resonating mass held in parallel relative to one another within the housing. The plurality of resonators may comprise a first resonating mass and a second resonating mass held and moveable within the first resonating mass. The tuned mass damping mechanism may be removably attached to the wearable base, and a plurality of tuned mass damping mechanisms may be removably attached to the wearable base.

One or more characteristics of the tremor in the outer extremity of the subject may be measured and recorded, for example the amplitude and frequency of the tremor(s). The measurement may be performed by a mobile and/or computer application coupled to the apparatus.

In another exemplary method, a wearable base to be worn over at least a joint of the outer extremity may be provided and movement of a distal moving region of the wearable base worn on the outer extremity relative to a proximal fixed region of the wearable base may be dampened in response to tremor movement in the outer extremity using a frictional damping mechanism. Movement may be dampened using the frictional damping mechanism by applying a force opposite in direction to the tremor movement in response to the tremor movement with the frictional damping mechanism. The wearable base may be worn over a wrist and at least a portion of a hand of the subject, and sometimes a distal forearm of the patient.

The force opposite in direction to the tremor movement may be applied by a viscoelastic material of the wearable base. The viscoelastic material may be configured to deform and interfere with the tremor movement in response to the tremor movement. The force opposite in direction to the tremor movement may be applied by at least one tension element held in tension within a body of the wearable base. An amount of tension of the at least one tension element may be adjusted.

One or more characteristics of the tremor in the outer extremity of the subject may be measured and recorded, for example the amplitude and frequency of the tremor(s). The measurement may be performed by a mobile and/or computer application coupled to the apparatus.

In another exemplary method, a wearable base to be worn over at least a joint of the outer extremity may be provided and movement of the outer extremity may be dampened using a tuned mass damping mechanism coupled to the wearable base worn on the outer extremity. Movement may be dampened using the tuned mass damping mechanism by providing a plurality of resonators held within a housing coupled to the wearable base and oscillating a plurality of resonating masses within the plurality of resonators. These resonator(s) may also include torsional pendulum(s) and/or other rotation resonators. The amount of oscillation allowed to at least one resonator of the plurality of resonators may be adjusted. The wearable base may be worn over a wrist and at least a portion of a hand of the subject, and sometimes a distal forearm of the patient.

The plurality of resonators may comprise a first resonating mass and a second resonating mass held in parallel relative to one another within the housing. The plurality of resonators may comprise a first resonating mass and a second resonating mass held and moveable within the first resonating mass. The plurality of resonators may comprise a first resonating mass and a second resonating mass held in series relative to one another within the housing. The plurality of resonators may comprise a first resonating mass and a second resonating mass held in series relative to one another within the housing.

The tuned mass damping mechanism may be removably attached to the wearable base, and a plurality of tuned damping mechanisms may be removably attached to the wearable base. Tremor parameters such as amplitude, intensity, and frequency may be tracked by the device and synced with a mobile and/or computer application. Information such as changes in amplitude and/or frequency of the tremor may be valuable to the user and their physicians. This data, for instance, may provide insight to the progression of the user's condition and/or if the type or dosage of medication needs to be changed.

One or more characteristics of the tremor in the outer extremity of the subject may be measured and recorded, for example the amplitude and frequency of the tremor(s). The measurement may be performed by a mobile and/or computer application coupled to the apparatus.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a side perspective view of a tremor dampening device worn over a wrist and a hand of a subject, according to many embodiments.

FIGS. 2A1-2A3 show section views of a tuned mass damping mechanism with parallel resonators, according to many embodiments, with FIG. 2A1 showing a perspective view and FIGS. 2A2 and 2A3 showing side views.

FIGS. 2B1-2B3 show views of a tuned mass damping mechanism with embedded resonators, according to many embodiments, with FIGS. 2B1 and 2B2 showing top-down section views and FIG. 2B3 showing an exploded view.

FIGS. 2C1 and 2C2 show top-down section and exploded views, respectively, of the tuned mass damping mechanism of FIGS. 2A1-2A3.

FIGS. 2D1 and 2D2 show magnified section views of a tension adjustment mechanism for a resonator of a tuned mass damping mechanism, according to many embodiments.

FIG. 2E shows a front view of a tuned mass damping mechanism attached to straps to aid in the wearing of the mechanism, according to many embodiments.

FIG. 3A-3C show another tuned mass damping mechanism, according to many embodiments, with FIG. 3A showing a perspective view of the mechanism as worn over a wrist and a hand of a subject, FIG. 3B showing a perspective, partially section view, and FIG. 3C showing an exploded view of the mechanism as worn over a wrist and a hand of a subject.

FIGS. 4A-4D3 show a frictional damping mechanism, according to many embodiments, with FIG. 4A showing a side view of the frictional damping mechanism as worn over a wrist and a hand of a subject, FIGS. 4B1 and 4B2 showing side section views, FIG. 4C showing a top section view, FIGS. 4D1 and 4D3 showing perspective section views, and FIG. 4D2 showing an exploded view.

FIGS. 5A-5C show side, top, and perspective views, respectively of a resonator in the form of one or more balls with circular ball transfer(s), according to many embodiments.

FIGS. 6A-6C show side, bottom perspective, and top perspective views, respectively of a resonator in the form of one or more balls with rectangular ball transfer(s), according to many embodiments.

FIGS. 7A and 7B show top and perspective, section views, respectively, of a system of embedded resonators arranged in series and parallel, according to many embodiments.

FIGS. 8A and 8B show perspective and side views, respectively, of a system of resonators arranged in series, according to many embodiments.

FIG. 9 illustrates a graph of a hand tremor amplitude response at different tremor frequencies, according to many embodiments.

FIGS. 10A-10M show rotational to linear motion mechanisms for coupling resonator(s) to a movable portion of a wearable base, according to many embodiments. FIGS. 10A-10I show slider-crank and hinge (SCH) mechanisms for coupling resonator(s) to the movable portion of a wearable base, according to many embodiments; FIG. 10A shows a side view of a SCH mechanism on the wearable base and worn on the hand and wrist; FIG. 10B shows a perspective, section view of the same; FIG. 10C shows a magnified view of the same; FIGS. 10D and 10E show side, section views of the same; FIG. 10F shows a perspective, section view of the same; FIG. 10G shows a top, section view of the same; FIG. 10H shows a perspective, section view of a SCH mechanism with an intermediary slider piece; and, FIG. 10I shows a top, section view of the same. FIG. 10J shows a side view of a SCH mechanism with torsional pendulum(s), the SCH mechanism being on the wearable base and worn on the hand and wrist; and, FIG. 10K shows a perspective view of the same. FIG. 10L shows a side view of a “Scotch Yoke” mechanism for coupling resonator(s) to the movable portion of a wearable base, and, FIG. 10M shows a perspective view of the same.

### DETAILED DESCRIPTION OF THE INVENTION

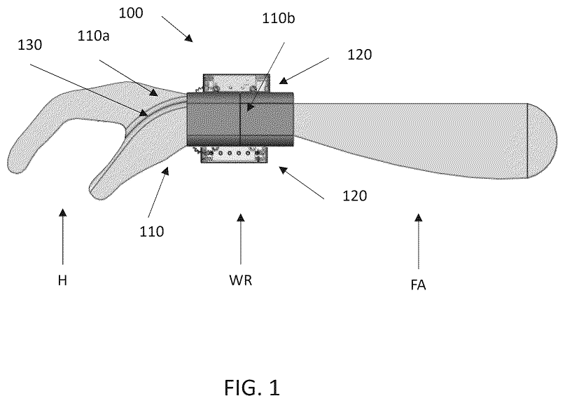

Disclosed herein are systems, devices, and methods to treat tremor in outer extremities by dampening tremor movement(s) of the outer extremities. Referring to FIG. 1, a wearable tremor dampening device 100 may comprise a wearable base 110 and use two mechanisms: (1) tuned mass dampers (TBD) 120 and (2) frictional damping (FD) via a frictional damping mechanism 130. The wearable base 110 may comprise a distal portion 110a configured to be worn over at least a portion of the hand H of the user and/or at least a portion of the wrist WR of the user and a proximal portion 110b configured to be worn over at least a portion of the wrist WR of the user and/or at least a portion of the forearm FA of the user. The TMD mechanism(s) 120 may be coupled to the proximal portion 110b of the wearable base 110. The TMD mechanism(s) 120 may be placed above and/or below the user's wrist WR and/or distal forearm FA. Alternatively or in combination, the TMD mechanism(s) 120 may be placed on either lateral side (i.e., left and/or right sides) of the user's wrist WR and/or distal forearm FA. The FD mechanism 130 may be located in at least the distal portion 110a of the wearable base, i.e., the half-glove wearable base 110 that covers part of the hand H and part of the wrist WR and may extend to the proximal portion 110b of the wearable base, i.e., the part of the base 110 over the distal forearm FA. In some embodiments, the TMD mechanism(s) 120 are detachable, so the user can choose to use only the FD mechanism 130 in the glove 110, the FD mechanism in the glove 110 and one TMD device 120, or the FD mechanism 130 in the glove 110 and two or more TMD devices 120. In some embodiments, only one or more TMD devices may be used with the half-glove (with no FD mechanism in the half-glove). In some embodiments, only the viscoelastic glove with no FD mechanism in the half-glove and no TMD devices may be used. The use of only the viscoelastic glove may be enough to damp tremors in users with sufficiently small tremor amplitudes. One or more TMD devices 120 may be placed above, below, left of, right of, and/or or otherwise around the user's wrist WR and distal forearm FA. In some embodiments, a plurality of TMD devices 120 may be worn around the user's wrist WR and/or distal forearm FA, as in a bracelet. At least some, if not all, of the TMD devices 120 may be curved to accommodate for the shape of the user's wrist WR and/or distal forearm FA.

### Tuned Mass Damper Mechanism.

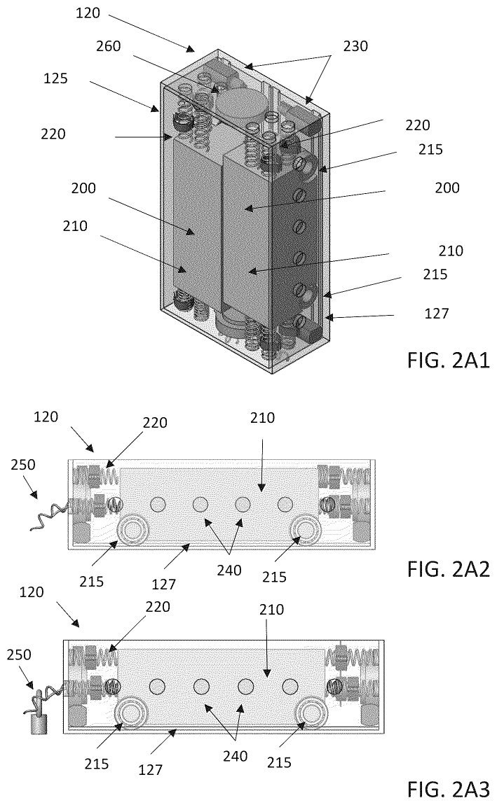

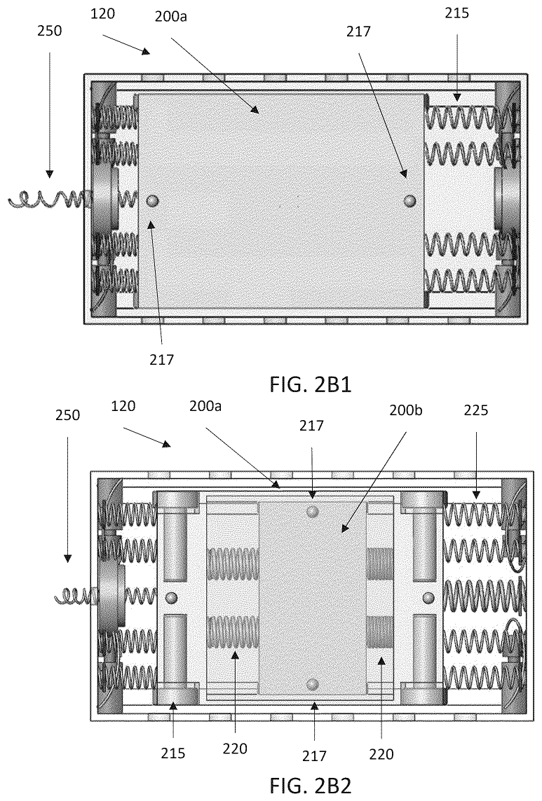

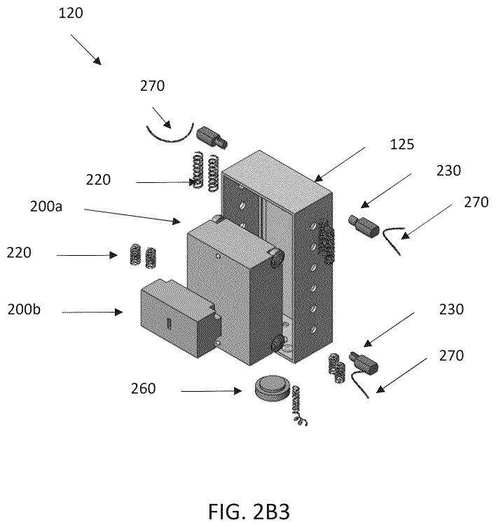

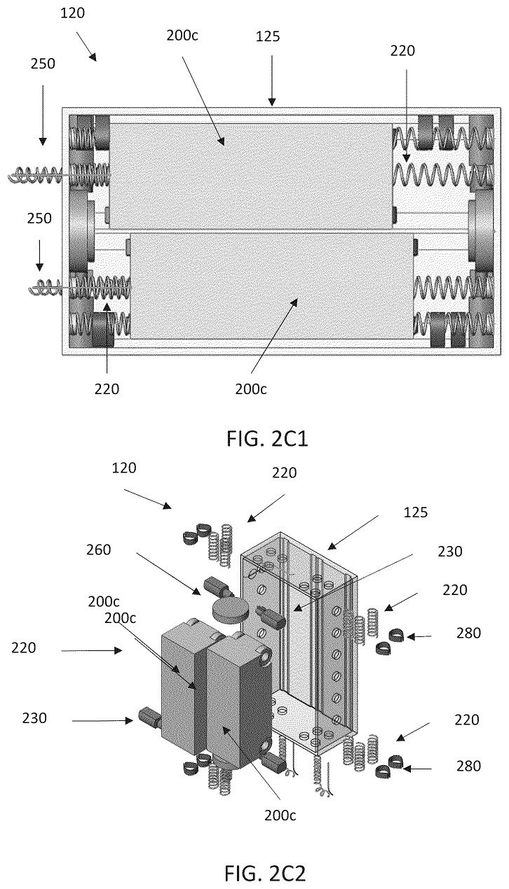

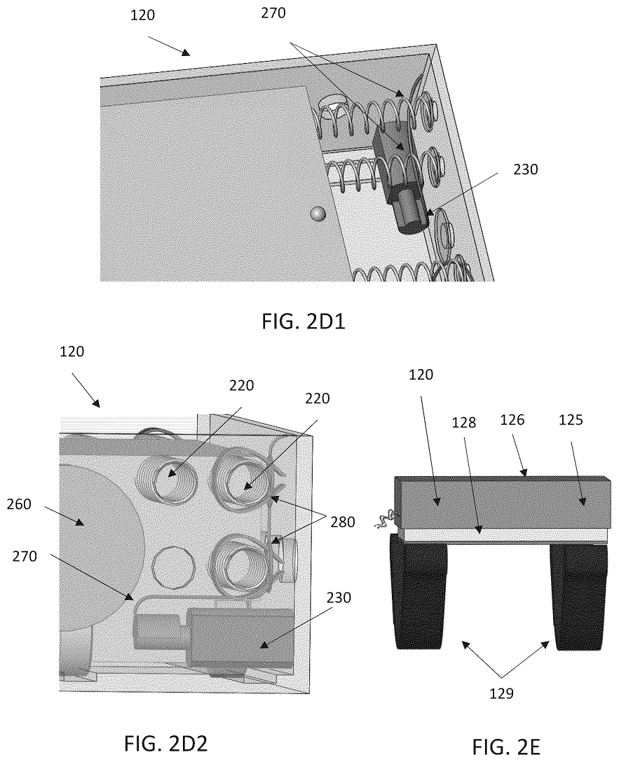

The tuned mass damper mechanism 120 comprises multiple mass-spring-damper systems or resonators 200 arranged inside the housing 125 of the tuned mass damper mechanism. A mass-damper system or resonator 200 may include a resonating mass 210 and spring(s) 210 coupled to the resonating mass 210 to facilitate oscillations of the resonating mass 210 within the housing 125. When users begin experiencing hand tremors, the resonating mass(es) 210 of the mass-spring-damper sub-systems or resonators 200 may oscillate such that they destructively interfere with the motion or movement of the tremor. The movement of the resonating mass(es) 210 of the resonators 200 may dampen the hand tremors and consequently may stabilize the hand of the patient or user. The tuned mass damper mechanism 120 may lie above, below, left of, right of, and/or otherwise around the wrist WR and distal forearm FA as shown in FIG. 1. In some embodiments, the tuned mass damper mechanism 120 may be slightly curved at the edges to take the form of the wrist WR of the user. Referring now to FIGS. 2A1-2A3, the housing 125 may include a track 127 upon which the resonating mass(es) 210 may travel back and forth. The track 127 and/or the housing 125 may be made of a rigid material or metal such as carbon fiber, fiber glass, aluminum, titanium, stainless steel, a metal alloy, or the like. The track 127 and/or housing can also be made of rigid plastics (e.g., high-density polyethylene, ABS plastic, and acetal). In some embodiments, the track 127 and/or housing may be made of a low-density material that the majority of the mass of the wearable device 100 can be located in the resonating mass(es) 210. This low-density material should also be rigid and strong enough to withstand impact forces (e.g., falling from a low height) without fracturing or deforming. The track 127 may be located at and/or formed on a bottom side of the housing 125. Thin ridges may be cut along the bottom of the track 127. One or more ball bearings 215 on the resonating mass(es) 210 may align with the location of these ridges, as the ridges guide the resonating mas(es) 210 along one direction. As an alternative or in combination with the ball bearings 215, roller bearings or simple spheres may be used. The thin ridges of the track 127 can prevent the resonating mass(es) 210 from sliding laterally or colliding with other components. The ridges and/or the track 127 may also be coated with a layer of synthetic rubber (typically a thin layer), noise dampening tape or material, and/or Teflon to reduce noise and friction. In some embodiments, the housing 125 and/or the track 127 are coated internally and/or externally with sound dampening, absorbing, and/or proofing materials (e.g., acoustic foam or panels, mass loaded vinyl, to name a few) to reduce potential noise from oscillation. To provide sound dampening, sound absorption, and/or soundproofing, one or more layers of dense sound proofing (e.g., industrial) blankets, pads, and/or rugs with a high STC rating, sometimes made of a polyester and/or cotton material (e.g., moving blankets), may be used. Alternatively or in combination, sound proofing sheets and/or boards such as mineral wool, soundproof fiberglass, and/or polyester absorption panels may be used. There may be a gap between these layers and the housing to create a layer of insulation, which can also aid in sound proofing. These layers (and/or additional layers of soft, flexible materials such as low density polyethylene, nylon, and synthetic rubbers) may also aid in protecting the device/housing from the moving resonator or other components inside (e.g., the resonator colliding against the sides of the devices during tremors or if the device is dropped).

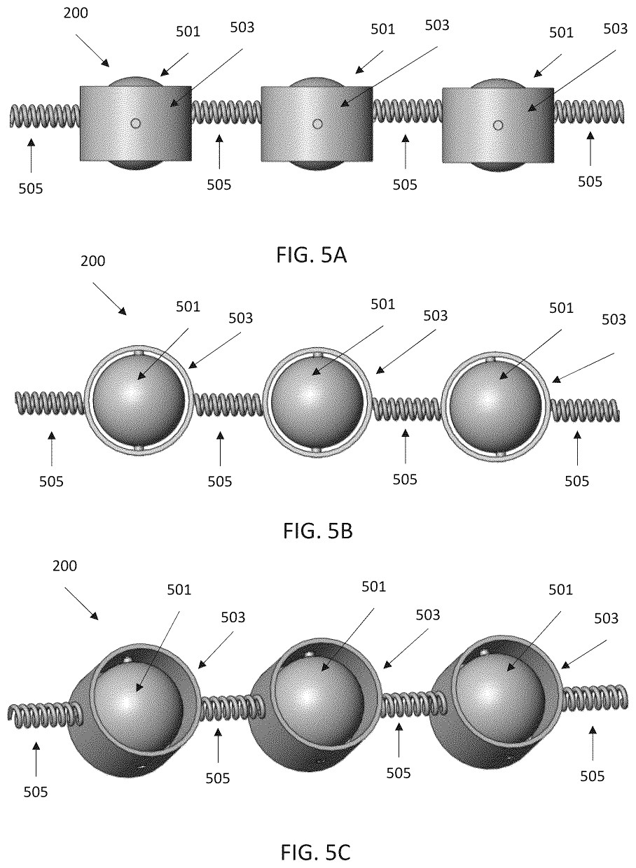

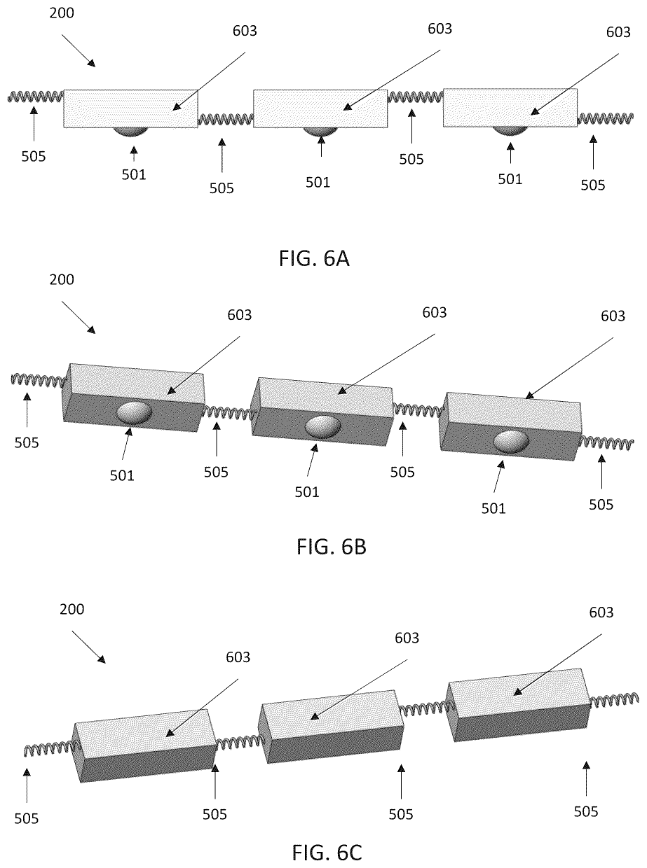

The resonating mass(es) 210 may be made of a high-density material or metal, for example, tungsten, lead, copper, nickel, iron, brass, and/or alloys of the aforementioned metals. In some embodiments, the resonating mass(es) 210 may be coated and/or enclosed in another material (e.g., lead interior with brass exterior). One or more springs 220 may be attached from the ends of the housing 125 to either side of the resonating mass(es), allowing the resonating mass(es) to oscillate within the housing 125. The spring(s) 220 may comprise linear spring(s), with a linear relationship between force and displacement. In some embodiments, these spring(s) 220 may comprise non-linear springs (e.g., conical, tapered, convex, concave, dual pitch). In some embodiments, these spring(s) 125 may comprise constant-force, extension, Volute, Drawbar, and/or Belleville springs. The ball bearings 215 may be attached to the bottom corners of the resonating mass(es) to allow low friction oscillation of the resonating mass(es) 210. The ball bearings 215 may be High ABEC stainless steel ball bearings, for example. Other suitable materials for the ball bearings 215 may include tungsten, chrome steel, steel alloy, iron, or combinations of the aforementioned, to name a few. In some embodiments, the outside of bearings/wheels may be coated and/or wrapped in a rubber-like material (e.g., synthetic rubbers, neoprene, silicone, nitrile, vinyl, neoprene, nylon) or noise damping tape. One or more lateral springs 240 may also be attached laterally from the sides of the housing 125 to the sides of the resonating mass(es) 200 to hinder sideways movements. The lateral spring(s) 240 may also act to create a tuned mass damper system in the lateral direction and damp tremors that may occur along that axis. The lateral springs may also be attached between the two or more adjacent resonators (in the case of parallel TMD devices, for example) for the same or similar purpose. The resonating mass(es) 210 may be shaped to fit within the housing 125 such that movement can be largely restricted to the desired directions for oscillation. For example, as shown in FIG. 2A1, the hollow interior of the housing 125 of the resonator 200 may be rectangular and the resonating mass(es) 210 may be shaped to fit with each other to form a complementary rectangular shape within the hollow interior, allowing more oscillating movement within the housing 125 while lateral movement is restricted. In some embodiments, the resonator 200 may comprise one or more balls 501 (e.g., metal ball(s)) which may be attached to a ball transfer 503. The dimensions of the ball transfer 503 may vary. Springs 505 may be attached from the sides of the housing 125 to the ball transfers 503. Springs 505 may also be attached between adjacent resonators. This attachment may allow the resonator 200 (including, the balls 501 and ball transfers 503) to oscillate both through linear motion and rotation. Allowing the resonator(s) 200 to both rotate and move linearly may allow the resonator(s) 200 to store more energy that the resonator(s) “absorb” from the tremor, which may further damp the tremor. Variations of resonator shapes are shown in FIGS. 5A-5C (circular ball transfers 503) and 6A-6C (rectangular ball transfers 603).

The side of the housing 125 closest to the hand H of the user may have small openings. Links 250 such as springs, metal wires, and telescoping connectors may pass through these openings and attach the resonator(s) 200 to the half-glove 110. In some embodiments, these links 250 pass through hoops to help guide the links between the resonator(s) 200 and glove 110. These links 250 (e.g., springs and wires) can transmit the force and movement of the hand tremors to the resonator(s) 200 in the tuned mass damping mechanism 120. The movement of the tremors may cause the resonating mass(es) 210 to move, and the springs 220 may tune the resonating mass(es) 210 to oscillate so that they destructively interfere with the tremors. As a result of the movements of the resonator(s), the links/springs 250 may exert forces on the hand H against that of the tremor; and, this exertion of forces may reduce the net force acting on the hand H, thus dampening the tremors. Some of the energy from the tremors may therefore be transferred to the oscillating resonating mass(es) 210. The user can have the ability to disconnect these links/springs 250 from the half-glove 110 if and when they choose to not use the TMD device 120. The user may then reconnect the links to the half-glove 110 when they wish to use the TMD device 120 again.

In some embodiments, there may be an intermediary slider piece between the hand and the resonator(s). The slider piece may rest on the device track and may be proximal to the hand (relative to the resonators). The slider piece may comprise a metal or plastic sheet/block on wheels/ball bearings, allowing the slider piece to also move along the device track as the resonator(s) would. The links, such as springs, metal wires, telescoping connectors, and/or even rigid links such as metal rods/bars may link the hand/glove to the slider. On the other side, horizontal springs may be attached from the slider to the resonator(s) in the device. In this way, the force of the tremor can be transmitted to the resonator(s) through the slider and links. For instance, when the hand deflects upward, the link between the glove and slider may first push the slider away from the hand, which may then exert a force on the resonator(s) causing the resonator(s) to oscillate in the TMD device. This slider may be located inside the TMD device. If located inside the device, the front side of the housing may have springs connected to the slider and/or stoppers to prevent the slider from colliding against the housing. In some cases, the slider may also simply be the front side of the housing (side proximal to the hand). This front side of the housing would then oscillate when tremors begin and also cause the resonator(s) to oscillate as they are connected together by springs. Spring guides may be used to keep the springs horizontal and stable, as described elsewhere herein.

One or more small steel ball bearings 217 may be attached to the top of the resonating mass(es) 210 and may be in contact with the roof of the housing 125, as shown in FIGS. 2B1 and 2B2. While small steel or metal ball bearings 217 are described, it is understood that simple metal or plastic balls, rollers, and/or wheels may be suitable as well. The ball bearings 217 may prevent the resonating mass(es) 210 from colliding with the roof when the housing 125 is flipped over and can allow for the resonating mass(es) 210 to oscillate smoothly, instead of undergoing high friction sliding along the roof.

The arrangement of the mass-spring systems 200 can differentiate the top and bottom TMD devices 120. Both may contain multiple resonators 200. In a first mass-spring system 130, at least one second, smaller resonator 200b lies inside the larger resonator 200a, as shown in FIGS. 2B1-2B3. The larger resonator 200a may act as the housing for the smaller resonator 200b inside it. Springs 220 are attached on either side of the smaller resonator 200b to the inside of the larger resonator, allowing the smaller resonator 200b to oscillate inside the larger resonator 200a. Similar to before, there may be ridges at the bottom of the inside of the larger resonator 200a to prevent the smaller resonator 200b from moving sideways. Lateral springs attached from the inside sides of the larger resonator 200a to the sides of the smaller resonator 200b may also help keep the smaller resonator 200b moving along one direction. This is referred to an embedded TMD system, where both the inner and outer resonators contribute to damp the hand tremors

In the other TMD device 120, there may be two mediums-sized resonators 200c working in parallel in the main housing 125, as shown in FIGS. 2C1 and 2C2. While shown with the same or similar sizes in FIGS. 2C1 and 2C2, the resonators 200c may be of different sizes relative to one another, and the resonator(s) 200c may be large, medium, and/or small sized. Each resonator 200c may be attached to its own set of springs 220, so the resonators 200c may move independently of each other. Depending on the effective spring constants of the respective springs, the movements of the resonators 200c can be same or vary at different times. The resonators 200c may also be attached separately to the glove by links 250 (e.g., springs, wires, telescoping connectors, etc.) through openings in the side of the housing 125 closest to the hand.

In some embodiments, resonators may be provided inside as in FIG. 2B1 and FIG. 2C1, making this at least an embedded TMD system. This configuration may allow the housing to contribute to the goal of damping the tremor, which may allow the device to be smaller and/or lighter.

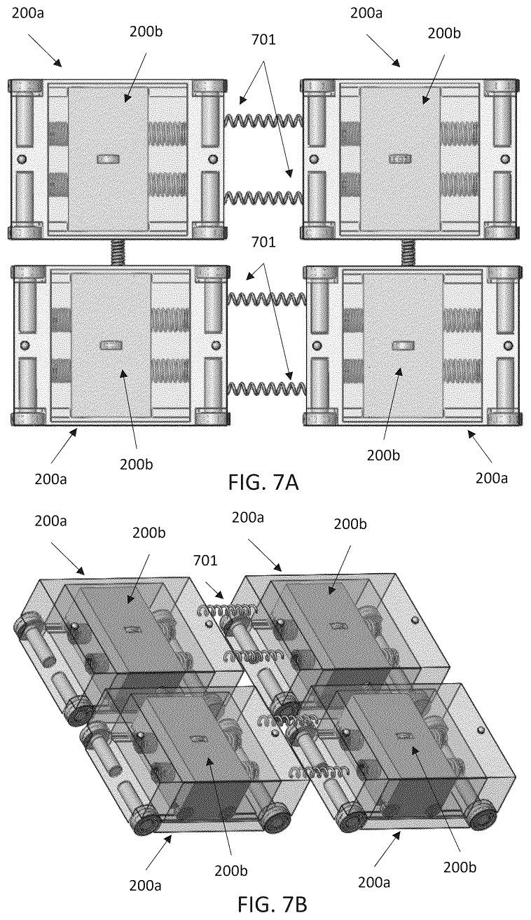

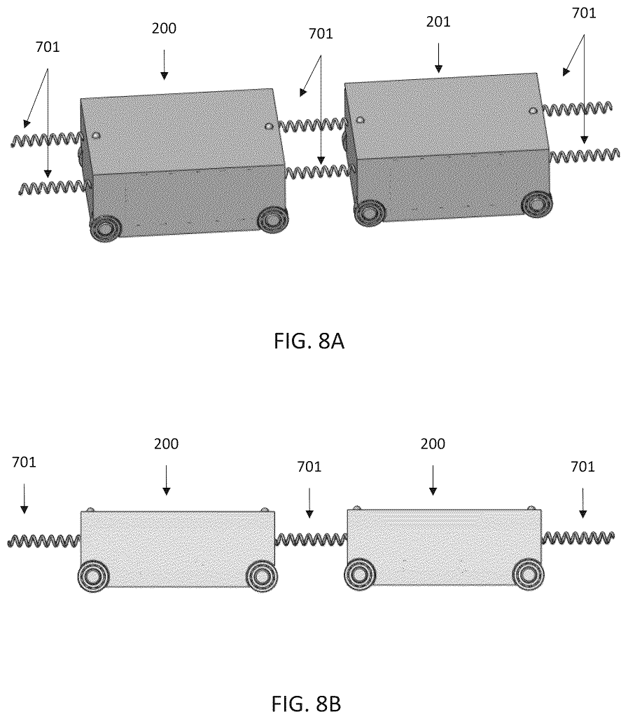

In some embodiments, the resonators 200 are arranged in series, as shown in FIGS. 7A-7B (in both series and parallel) and 8A-8B (in series). There may be multiple resonators 200 between the two ends of the housing, which are attached to each other by internal springs 701, as shown in FIGS. 7A and 7B. Their movements may therefore affect one another (they do not move independently of one another). Springs may be attached from one end of the housing to the resonator 200 proximal to it (and similarly on the other end of the housing). Internal (lateral) springs 701 may link the resonators 200 together. When tremors begin, the movement of the hand H can force the resonator 200 proximal to it to oscillate, which can induce the other resonators 200 in series to oscillate as well. Similar to the other TMD mechanisms, this mechanism can also increase the range of tremor frequencies for which the device is effective. In some embodiments, the tuned mass damper device 100 may comprise combinations of parallel, embedded, and/or series resonators. For instance, one 8-resonators system may comprise resonators 200a in parallel and two resonators 200a in series, each of which has a smaller resonator 200b embedded within, as shown in FIGS. 7A-7B. In some embodiments, the housing of the device can act as the outer resonator. The springs from the half-glove 110 may be attached directly to the housing 125 and the housing 125 may oscillate linearly along the extended half-glove 110 that it is affixed to. In some embodiments, the housing of the TMD device may also function as a resonator. Springs may be attached from the glove directly to the front side of the housing (for example, instead of to the internal resonators), allowing the housing to oscillate back and forth when tremors begin. Resonators may or may not be provided inside the housing in this scenario (if resonators are provided, the system effectively becomes an embedded TMD system). This configuration may allow the housing to contribute to the goal of damping the tremor, which may allow the device to be smaller and/or lighter.

The use of both embedded resonators 200a, 200b and parallel resonators 200c in the device 100 can account for the variability in the frequency and movement of the hand tremors. Patients or users may experience variation in their tremor frequencies and amplitudes within and between tremor episodes. These mechanisms can allow for a wider range of frequencies for which the devices are effective. In some cases, the TMD devices on the sides of the hand may be set up such that the resonators oscillate vertically instead of horizontally as shown in the previous figures. This oscillation could be through vertical linear motion or vertical rotation (where the proximal end of the resonator is fixed and the distal end rotates vertically). The springs in this case may be attached vertically as well to aid this oscillation.

While top and bottom TMD devices 120 are described, it is understood that various other arrangements of TMD devices 120 (for example, any combination of TMD devices 120 at direction(s) left, right, down, up, and combinations thereof) are contemplated. In some cases, neither a parallel system nor an embedded system is provided (i.e., there may be only one resonator inside the device). In some cases, combinations of parallel-series-embedded systems may be provided.

Another important part of the TMD devices 120 can be their ability to actively account for this hand tremor variation by changing the stiffness of the springs 220 acting on the resonators 200. Multiple mechanisms may be used to achieve this selective variation. A wire, belt, or rope 270 (hereinafter, referred to as wire 270) that pins back part of the spring 220 may be tightened to restrict the movement of a certain section of coils, as shown in FIG. 2D1. A strap 280 that is wrapped around the spring 220 may be tightened to restrict the movement of a certain section of coils of the spring 220, as shown in FIG. 2D2. In both mechanisms, when a certain section of the coils of the spring 220 cannot move, its effective length may decrease, and the stiffness of the spring may increase. Likewise, when the restriction is removed, the stiffness of the spring 220 may return to its original, unrestricted stiffness.

Both mechanisms may use a system of micro-motors 230 to restrict this movement. While micro-motors are described herein, other types of actuators may be used alternatively or in combination, such as linear actuators, rotary actuators, and/or other tools and devices that can toggle between two or more positions. The TMD device 120 may include an attached accelerometer. Referring to FIG. 2D2, when the attached accelerometer detects a change in frequency of the tremor, a specific pattern of motors may activate and rotate the wire(s) 270 and/or belt(s) 280 that are attached to it, thus tightening them. A mathematical model may inform which motors 230 are activated and when. In the first mechanism, the wires/belts 270 may be placed such that they pass through specific parts of the springs 220, as shown in FIG. 2D1.

When the motor 230 rotates and pulls the wire 270, the wire 270 may tighten and restrict the movement of coils proximal to the edges of the housing 125. This can effectively change the stiffness of the springs 220 and consequently may affect the movements of the resonators 200. The resulting movement of the resonators 200 may be better tuned to interfere with the new movement of the tremor. If the tremor frequency changes again, a different pattern of motors 230 may be activated and/or deactivated to better counter the new tremor movement. The wires 270 may be coated in a high-friction material (e.g., rubber) against the springs for better grasp of the coils and protection of the wires 270. Other types of suitable high-friction materials include synthetic rubber, thermoplastic rubber, nylon, polyvinyl chloride, semi-rigid PVC, neoprene, silicone, PTFE, and thermoplastic elastomers. These coatings can also aid in thermal and/or electrical insulation. The motors 230 may be powered by small, rechargeable batteries 260 inside the device 120.

In the second mechanism, the motors 230 may once again be attached to the wire, belt, or rope 270 that tightens when the motor 230 rotates in one direction, for example, if and when commanded in response to a tremor frequency change. The wire 270 may pass through and may be wrapped around multiple straps 280 that envelop a section of the spring 220, as shown in FIG. 2D2. Initially, the strap 280 may be loosely wrapped around the spring 220, in that the strap 280 may have little to no effect on the stiffness of the spring 220 just yet. If and when the accelerometer detects a change in tremor frequency, the appropriate motors 230 may rotate and pull the wire 270, causing the strap 280 to tighten around the spring 220. When the strap 280 is tightened, the coils under the strap 280 may be prevented from moving as a result of the high normal and frictional force acting on it. Similar to the previous mechanism, this tightening can change the effective spring constant acting on the resonators 200. Depending on which motors 230 are active versus inactive, many different effective spring constants can be achieved. In some cases, a variable stiffness spring system can be implemented using variable fluid damping methods. In some springs, an adjustable fluid damper may be placed between the springs and the sides of the housing. The amount of damping can affect the effective spring constant. In a two spring-system, for instance, at high damping values, the effective spring constant may simply be the sum of the stiffness of the two springs. At lower damping values, the effective spring constant may decrease. As tremor frequency changes, the fluid damping, and thus the effective spring stiffness, can be adjusted to best damp the tremor.

Another mechanism that may be used to vary the effective spring constant is one that controls the number of springs acting on the resonators. This can be achieved in a number of ways. The springs which are to be engaged may first be attached to the resonators on one side. On the other side, the springs may be connected to motors or actuators through a wire, cable, string, or rope (henceforth called “cable”). The motors or actuators may be located at the sides of the housing. The cable may initially be loose and the spring may not be engaged at this point. When there is a need to engage the spring, the motor or actuator may rotate or move such that the cable tightens. Once tightened, one end of the spring may remain attached to the resonator, while the other end may be fixed at the motor's location near the side of the housing. This may engage the spring, such that the movements of the resonator may cause the spring to stretch and compress; this may in turn affect the oscillation of the resonator. To disengage the spring, the motor may rotate in the opposite direction to loosen the cable. While motors and actuators are described herein, other types of actuators may be used alternatively or in combination, such as linear actuators, rotary actuators, and/or other tools and devices that can toggle between two or more positions. Spring guides may be used to keep the springs stable. Other methods to engage springs by pulling on or attaching one spring end to the resonator and/or housing may be employed.

Another mechanism that can be employed is a screw-slider-crank-like mechanism in which there are horizontal and vertical sliders that may be linked together by a connecting rod. The horizontal slider may be located along the sides of the housing. The vertical slider may move along the length of the spring and may restrict movements of the coils proximal to housing side from the point at which the slider is located. The vertical slider may be connected to the housing through telescoping connectors. Motors and/or linear actuators may be coupled to the horizontal sliders. The motors and/or linear actuators may move the horizontal sliders forwards and backwards. This may in turn move the vertical sliders upwards and downwards along the springs and may change the number of coils proximal to the housing sides and the effective spring constant. In this way, the effective spring constant acting on the resonators may be controlled. Multiple effective spring constants may be achieved depending on the location of the vertical slider.

Several other mechanisms can be used such as “antagonistic controlled stiffness” springs systems. For instance, pairs of springs may be connected to each other on one end by wrapping around pulleys. These pulleys may be coupled to the resonators. On the other end, the pair of springs may be connected to other springs and/or actuators that can pre-compress or pre-stretch the pair of springs. This pre-load may affect the force the springs exert on the resonators and the hand. This mechanism may be tuned by changing the amount of pre-load on the springs. Similar variations of such antagonistic controlled stiffness springs systems may be employed.

These spring stiffness control mechanisms may be used alternatively or in combination with one another.

Referring back to a mathematical model for motor or actuator activation, the mathematical model can determine which configuration of the mass-spring-damper system would most effectively dampen the tremor movements at a given moment in time. The model may use input measurements such as the masses of the resonators and the frequency of the tremors to determine the effective spring constant that would best counteract and reduce the amplitude of the tremoring hand. Tremor frequency can change during a tremor episode and/or between episodes. When the tremor frequency changes (e.g., a state change), a new mass-spring-damper system configuration may be needed to best damp the tremors in this new state. It may be impractical to change the resonator masses to achieve this new ideal configuration. Thus, the effective spring constants of the springs may be altered to account for this frequency change. The mathematical model can determine the ideal effective spring constants in this new tremor state and the motors achieve these new effective spring constants using the aforementioned mechanisms.

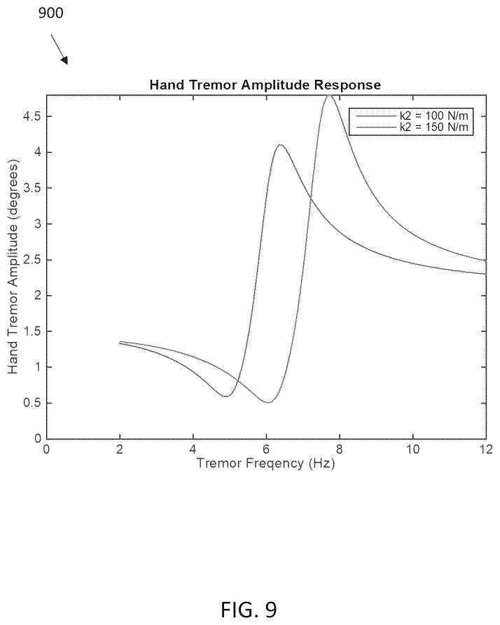

FIG. 9 illustrates a graph 900 of a user's hand's response to a specific tuned mass damper system configuration at different tremor frequencies. Given system parameters such as resonator mass(es) and damping constant(s), the model can calculate the hand's resulting amplitude at different tremor frequencies. The goal is to implement a mass-spring-damping configuration that can minimize the hand's amplitude (indicated by the dips in the graph). In this specific example, if the user's tremor has a frequency of 5 Hz, an effective spring constant of 100 N/m may be ideal to counteract the tremors. If the tremor frequency changes over time and increases to 6 Hz (20% increase), then the model indicates that an effective spring constant of 150 N/m may best counteract the tremors. These measurements and calculations can inform the device, and the device can thus implement the ideal effective spring constant whenever possible.

In some embodiments, the TMD device 120 may be used even without charging the battery 260. The motion of the resonator 200 is caused by the motion of the tremors, and thus it may not be necessary to have this device 120 charged at all times. When charged, the TMD device 120 can have the ability to calibrate or tune itself according to variations in the tremor frequency of the patient or user and thus be more effective. However, even without this automatic tuning, the TMD device 120 may still be effective in damping tremors, though the ability to account for frequency variations may be limited in some cases. When charged, the TMD device 120 may also collect data about the tremor frequency and strength, thus indicating to patients or users how their tremors, and by extension their conditions, are progressing over time. This information can be useful for both the patient and their doctor to help evaluate the progress of their condition and the need to change medication and/or dosage.

In some embodiments, the top cover 126 of the housing 125 can protect the inside contents of the TMD device 120, as shown in FIG. 2E. In some embodiments, the top cover 126 can connect to the remainder of the housing using snap-fitting tabs and/or screws. The cover 126 may comprise rigid materials like metals (e.g., aluminum, steel, titanium) and/or rigid polymers/plastics (e.g. PVC, acrylic), which may have a relatively low density to keep the overall mass of the device low, a high rigidity to prevent device fracturing or deformation, and/or weather-proof the device.

In some embodiments, the housing 125 includes a wrist-interface layer 128 between the metal and the wrist of the patient or user. This layer 128 can ensure a stiff connection between the track 127 and the forearm of the patient or user and can add a degree of comfort for the user. First, a rigid foam may be placed below the track 127 to ensure a stiff connection between the track 127 and the forearm. A layer of breathable rubber material (e.g., neoprene, nylon, polyester, cotton fabric, linen, silk, merino wool) can then be wrapped around this rigid foam to provide comfort to the patient or user. This layer may be in direct contact with the wrist of the patient or user. In some embodiments, only a breathable rubber material (e.g., neoprene, nylon, polyester, cotton fabric, linen, silk, merino wool) is used as the wrist-interface layer. In some embodiments, the wrist-interface layer comprises the same material as the half-glove 110, and may be an extension of the half-glove 110 to the area underneath the TMD devices 120 and above the distal forearm FA and/or wrist WR. In this embodiment, the device can counteract tremors that cause the patient's hand to rotate about the “rolling axis”, or the axis that passes through the middle of the wrist and the middle of the middle finger.

Finally, the devices 125 can be tightened to the wrist or forearm using detachable, hook-and-loop straps 129, as shown in FIG. 2E. These straps 129 can offer a sleek appearance, a continuous range of attachment, and/or a high degree of comfort. The TMD devices 125 can also be attached to an extension of the half glove 100 if straps are not desired, as shown in FIG. 1.

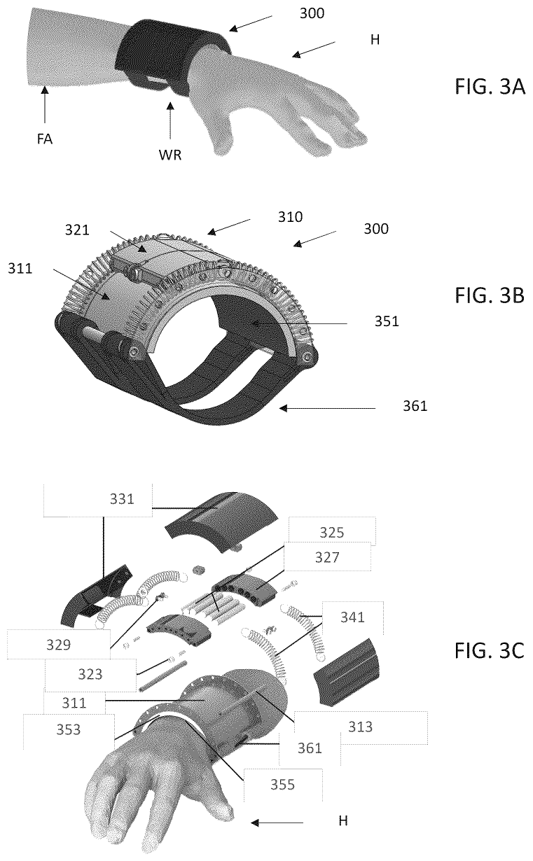

FIGS. 3A-3C show a further embodiment of a wearable tuned mass damper (TMD) mechanism or device 300. The TMD device 300 may be in a form factor of a lightweight bracelet-like device. The bracelet-like device 300 may wrap around the wrist WR of the user, as shown in FIG. 3A, and may use tuned mass dampers to dampen the tremors that ultimately stabilizes the hand, similar to the TMD device 125 described above. The TMD device 300 may comprises a mass-spring-damper system 310 inside the bracelet-like device. When tremors begin, the mass 320 inside the bracelet-like device 300 may oscillate such that it destructively interferes the tremor motion. The bracelet-like device 300 can have a circular form factor so that it can be easily worn around the wrist WR.

Referring to FIGS. 3B and 3C, the device 300 may comprise a track 311, a resonating mass 321, a cover 331, springs 341, a wrist interface layer 351, and straps 361. The top half of the bracelet-like device 330 may comprise a (metal, e.g., aluminum) track 311 shaped as a hemisphere. On either end of the track 311, there may be a strap connection rod 313. These rods 313 can connect the straps 361 on each side to the track 311, as well as secure the springs 341 to the track 311. There may be thin ridges that run along either side of the track 311, which can allow the track 311 to interface with the mass 321 because the ridges guide the mass 21 along one direction. As a result, the mass 321 can be prevented from sliding laterally or colliding with other parts of the device 300.

The resonating mass 321 may be made of two separate parts 327, each made from a metal such as brass, lead, copper, nickel, iron, and/or alloys of these and other metals, connected to one another with one or more frame connectors 329. The exterior may be coated/enclosed in another material (e.g., lead interior with brass exterior). The mass 321 may travel along the track 311 using ball bearings 323, for example, ¼ in. OD, ABEC-7 stainless steel ball bearings. These bearings 323 may be pressed onto pins, for example, stainless steel dowel pins, which may then be pressed into the holes on each corner of the subassembly of the mass 321. The mass components 327 may feature recesses for the bearings 323 and clearance for the dowel pins such that the bearings 323 do not extend beyond the mass face. To increase the overall density of the mass 321, holes, e.g., six 6 mm diameter holes, may be carved in the rods 325 (e.g., made of high-density materials such as brass and high-density tungsten or tungsten carbide) which may be inserted in these cavities. These rods 323 may be sandwiched between the two mass components 327. Fixture holes on the ends of these cavities may be used as tapped holes for set screws so that these rods 323 can be secured without press-fits. This lack of press-fits can allow the rods 323 to be easily removed. These tapped holes can also allow for the attachment of springs 341, wo on each side of the mass 321.

The cover 331 may protect the inside contents of the bracelet-like device 300. The cover 331 may, for example, comprise three high-precision 3D printed parts that connect to the track 311 via snap-fitting tabs that insert into the track flange holes. These snap fits can allow the cover 331 to be placed on or taken off easily.

A plurality of linear springs 341 (for example, four as shown in FIG. 3C) may be attached to the mass 321 on either end and may interface with the ends of the track 311. The radial force exerted by these springs 341 on the mass 321 may keep the mass 321 held onto the track 311. Depending on the tremor frequency, these springs 341 may be varied to achieve the appropriate spring constant, e.g., the diameter, length, coil density, thickness, and material of the springs may be chosen accordingly.

The wrist interface layer 351 comprises the layer between the track 311 and the wrist WR of the user. The goal of this layer 351 may be to ensure a stiff connection between the track 311 and the forearm of the wearer, as well as to add a degree of comfort for the wearer. A rigid foam 353 may be placed below the track 311 to ensure a stiff connection between the track 311 and the forearm of the wearer. Polyurethane foam may be used here because of its low density and high rigidity. A softer second layer 355, e.g., made of neoprene, can then be wrapped around this rigid foam layer 353 because of the breathability and comfort it can provide to the user. This layer 355 may be in direct contact with the wrist WR of the user.

As shown in FIG. 3A, the TMD device 300 may be strapped to the wrist WR with straps 361, which may comprise two hook-and-loop fluoroelastomer straps. The straps 361 can offer a sleek appearance, a continuous range of attachment, and a high degree of comfort. Alternatively or in combination, the TMD device 300 may be coupled to a wearable base 110 as described above. A benefit of having the mass-spring system 300 on the top half of the wrist WR and the straps 361 on the bottom half is that it can make the TMD device 300 more accessible to the user. The TMD device does not have to conform precisely to the shape and size of the wrist WR of the user, as it would have to if the tuned mass damper device 300 operated on the entire wrist WR. Accordingly, far fewer iterations of the TMD device 300 would need to be manufactured to supply the vast majority of tremor patients. And, the TMD device 300 can be customized easily and cost effectively. Tremors can be difficult to address because each patient has their own unique tremor movement and tremor frequency. The TMD device 300 can be customized to the specific frequency of a characteristic tremor of the user, which usually falls between 3-12 Hz. Custom assembly can be straightforward due to the modular design. This modular design can also make removing and replacing the components of the bracelet easy, should it need repair or recalibration. By determining the tremor frequency of the patient or user, a mathematical model can be provided to calculate the optimal mass-spring-damper system as described above.

The TMD device 300 may incorporate embedded tuned mass dampers similar to the TMD device 125 described above with respect to FIGS. 2B1-2B3, that is, another mass-spring system that is provided inside the mass 321. The smaller mass-spring system may be similar to the mass 321 and may include rods 325 which may be allowed to oscillate within the mass components 323 that are coupled to one another to form an enclosure defining the smaller mass-spring system. Springs may be provided at the ends of each of the rods 325 to facilitate such oscillation. When the tremor begins, the mass 321 inside the bracelet-like device 300 and the smaller mass inside the larger mass 321 can begin to oscillate in a way that destructively interferes with the tremor motion. By tuning both mass-spring systems to the patient's tremor frequency, the bracelet-like TMD device 300 can better reduce hand tremors over a broader range of frequencies. For example, if one mass-spring system is tuned for patients with a first tremor frequency, e.g., 3.8-4 Hz, then a system with a further embedded mass-spring system may work well for patients with over a broader tremor frequency spectrum, e.g., in the range of 3.3-4.5 Hz.

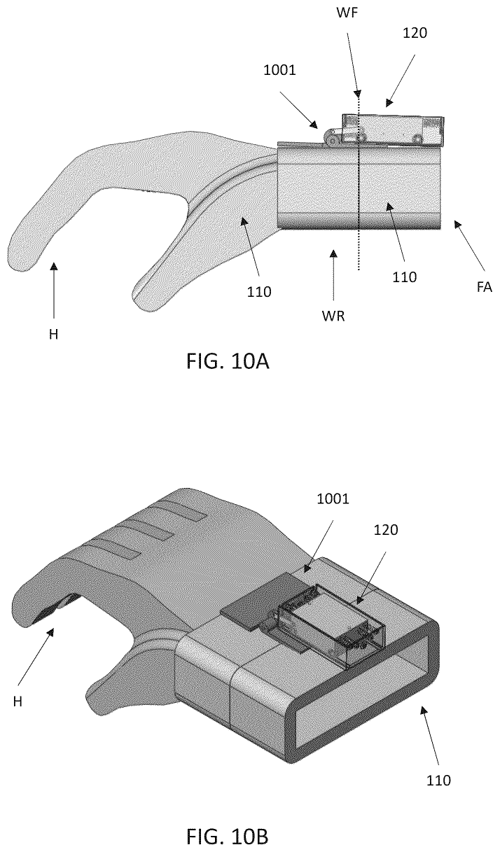

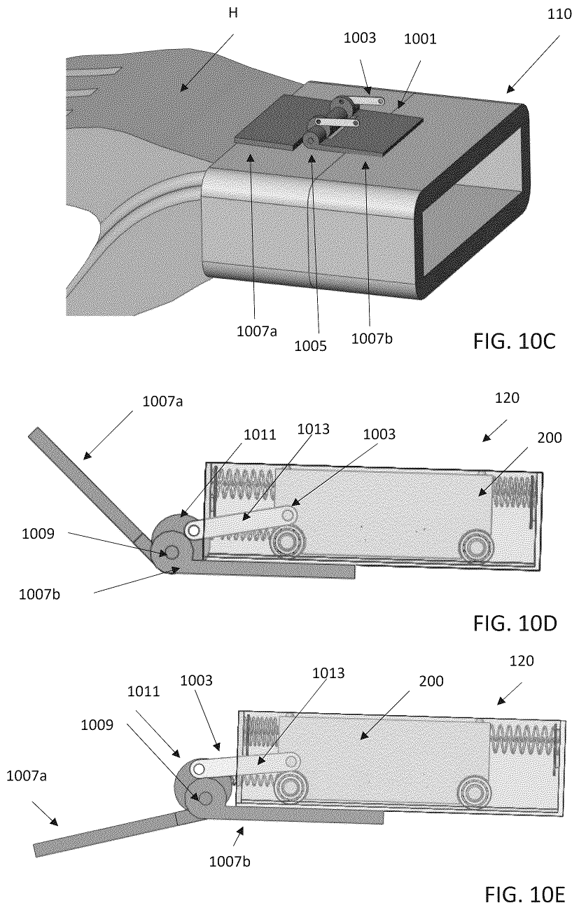

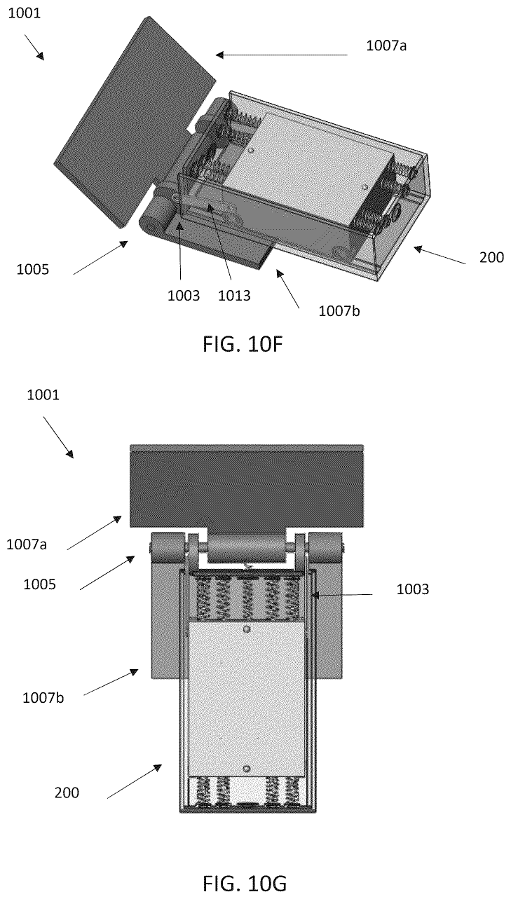

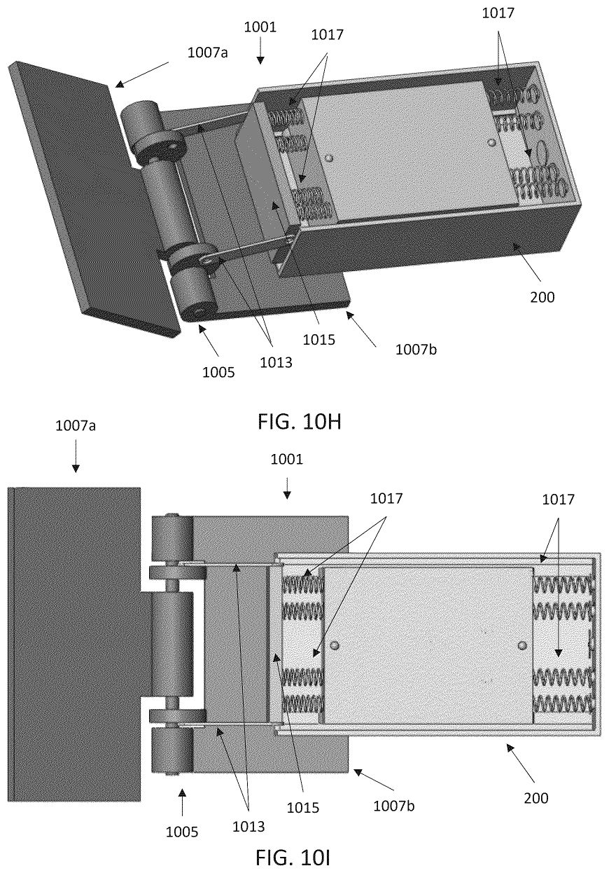

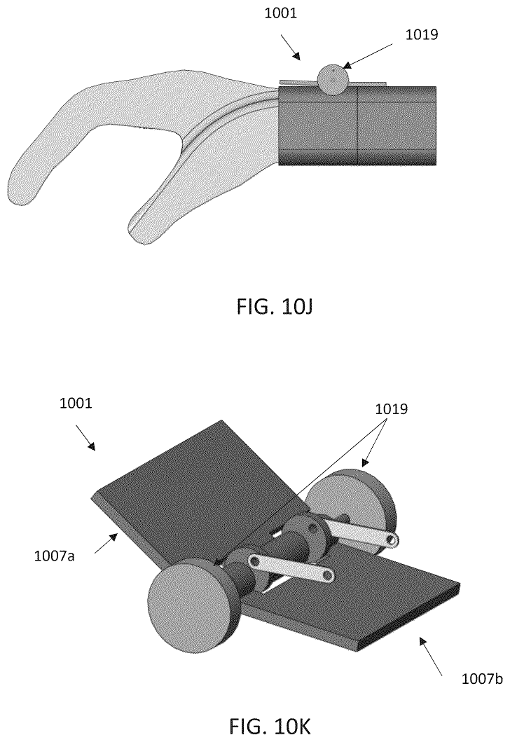

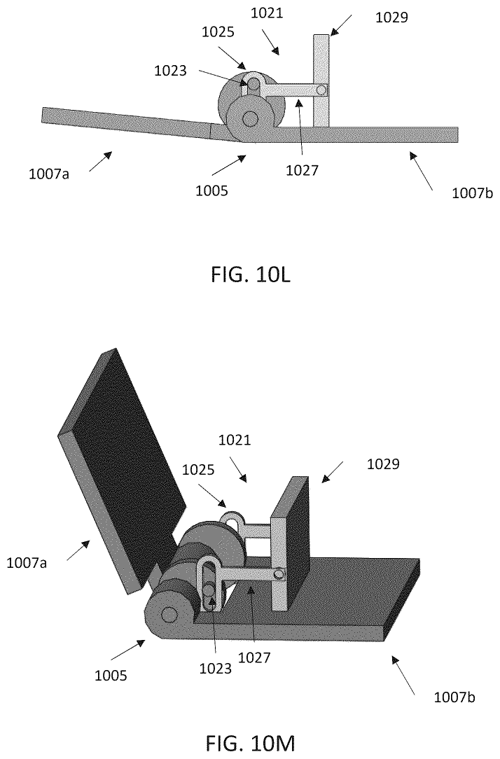

In some embodiments, the link between the TMD device 120 and the hand H may comprise a slider-crank-like mechanism 1003 attached to a hinge mechanism 1005 (henceforth referred to as SCH mechanism 1001), as shown in FIGS. 10A-10K. This SCH mechanism 1001 is comprised of two hinge pieces 1007a, 1007b, which form the hinge mechanism 1005 and are connected by a rod 1009 passing through them. The hinge mechanism 1005 may lie on the wrist flexion WF axis such that the center of the hinge mechanism 1005 can remain stationary even when hand tremors begin. One hinge piece 1007a overlays the part of the glove 110 that covers the hand H, while the other hinge piece 1007b overlays the part of the glove 110 that covers the wrist WR and/or distal forearm FA. The former piece, also referred to as the moving piece 1007a, can rotate up and down in accordance with the tremoring hand. The latter piece, also referred to as the fixed piece 1007b, may be fixed in place over the wrist WR and/or distal forearm FA, and does not move in relation to the tremoring hand. During a tremor, when the hand H moves upward, the moving piece 1007a can deflect upward as well. The rod 1009 connecting the hinge mechanism 1005 and typically affixed to the moving piece 1007a can also rotate accordingly. This rod 1009 can also be connected to a slider-crank-like mechanism 1003 as shown in FIG. 10D. During a tremor episode, when the hand H moves upward, for example, the moving piece 1007a may deflect upward as well. The rod 1009 connecting the hinge mechanism 1005 and affixed to the moving piece 1007a can also rotate accordingly. This rod 1009 may also be connected to the slider-crank-like mechanism 1003. When the hand H and the moving piece 1007a move upward, the rod and crank-like piece 1011, which is attached to the rotating rod 1009, may rotate clockwise. One or more other connecting rods 1013 may be attached from the crank 1011 to the resonator 200 and/or its housing in the tuned mass damper system 120 as shown in FIGS. 10C-10E. Because the resonator 200 and/or its housing are configured to move linearly, the connecting rod 1013 can push the resonator 200 away from the hand H when the hand deflects upward (FIG. 10D). Likewise, when the hand H deflects downward, the connecting rod 1013 can pull the resonator 200 towards the hand H (FIG. 10E). In this way, the external force of the tremor can be transmitted from the hand H to the resonator 200 and/or its housing. The resonator 200 and/or its housing may oscillate as a result of the external force and the springs attached on either side of the resonator 200. As described earlier, the oscillation of the resonator 200 can destructively interfere with the motion of the hand tremors. The resonator 200 can exert a force on the hand H through the same mechanism by which the hand exerts a force on the resonator, the SCH mechanism 1001 as previously described. The SCH mechanism 1001 may also be connected to the resonator 200 and/or its housing through links such as wires, springs, and telescoping connectors, as described earlier. In addition to the slider-crank-like mechanism 1001, wires, springs and telescoping connectors may be attached from the resonator 200 to the moving piece 1007a of the SCH mechanism 1001; these additional structure(es) may further aid in force transmission. This SCH mechanism 1001 may overlay or be embedded in the half-glove 110. The SCH mechanism 1001 may be located on the top and/or bottom of the half-glove 110, depending on where the TMD devices 120 are located. In some embodiments, the connecting rod 1013 is attached to a slider 1015 that also rests on the device track and is proximal to the hand (relative to the resonator(s) 200) as shown in FIGS. 10H-10I. The slider 1015 may then be linked to the resonator(s) 200 through horizontal springs 1017. Thus, when the hand deflects upward, the connecting rod 1013 may first push the slider 1015 proximally (shown in FIGS. 10H-10I as to the right), which may then exert a force on the resonator(s) 200 causing the resonator(s) 200 to oscillate in the TMD device. The slider 1015 may also have ball bearings to aid in its horizontal movement along the track for the resonator(s) 200. This sider 1015 may be located inside the TMD device. The slider 1015 may also simply be the front side of the housing (side proximal to the hand), which oscillates. In some cases, the connecting rod 1013 is attached directly to the springs that are attached to the resonator(s) 200. Spring guides may be used to keep the springs horizontal and stable.

In addition to the slider-crank-like mechanism described above, other similar rotary-to-linear-motion mechanisms can be used to achieve this force transmission between the hand and the TMD device. For instance, a ‘sun and planet gear’ mechanism can be used as this linkage. Gears may be attached to the hinge in place of the crank that is shown in FIGS. 10A-10K (henceforth called ‘hinge gears’). Another set of gears (henceforth called ‘outer gears’) may then be linked to the hinge gears, such that when the hand moves upward, the hinge gears rotates clockwise (as seen from the perspective of FIG. 10A) and the outer gears rotates counterclockwise. The two sets of gears can remain tangent and linked to one another through a connecting rod. Also attached to the axle of the outer gear may be a beam that is attached on the other end to the resonator(s) or a slider that is then connected to the resonators by springs. When the hand deflects up, the hinge gear may rotate clockwise and the outer gear moves along the hinge gear in a clockwise direction as well. The beam may then exert a force on the slider, which rests on the device track proximal to the hand and pushes it proximally. The slider can therefore exert a force on the resonator(s), through the internally attached springs, which may cause the resonators to oscillate. Thus, the movement of the tremor may exert a force on the TMD system, and the movement of the resonators, which may destructively interfere with the oscillation of the hand tremors, may transmit the force to damp the tremors through this same mechanism.

In some embodiments, torsional pendulums 1019 are attached to the ends of the rod 1009 that connects the hinge pieces 1007a, 1007b, as shown in FIGS. 10J and 10K. In some embodiments, there may be an intermediary link between the rod 1009 and the torsional pendulum 1019 (e.g. another rod or wire). The rotation of the rod 1009 during tremors may cause the torsional pendulums 1019 to rotate as well. This may introduce torsional damping, as the torsional pendulums 1019 may resist and counteract the tremor rotation, thus damping the tremor). The torsional damping may depend on the torsional spring constant, which may be designed to counter tremors in the relevant 3-12 Hz frequency range. Torsional dampers may also be attached to this mechanism to introduce further damping in the system when tremors occur. The rod 1009 connecting the hinge pieces 1007a, 1007b may be coated in rubber-like materials (e.g. synthetic rubber, nylon, silicone, semi-rigid PVC).

Another rotation-to-linear-motion mechanism that can be used is the “Scotch Yoke” mechanism 1021, as shown in FIGS. 10L and 10M. Similar to the embodiments shown in FIGS. 10A-10K, a pin 1023 may be attached to the outer sides of the cranks on the hinge 1005. The pin 1023 may be fit into a yoke 1025 such that it is able to freely slide vertically along the yoke 1025. A beam 1027 may be attached to the yoke 1025 and can slide horizontally based on the movement of the yoke 1025. Attached to the other side of the beam 1025 may be a slider 1029 that can be attached to the resonator(s) through internal springs. During tremor episodes, when the hand moves upward, the hinge 1005 and pin 1023 may rotate clockwise. The yoke 1025 and beam 1027 may move to the right, and thus exert a force on the slider 1029, pushing it proximally as well. The slider 1029 may therefore exert a force on the resonator(s) through the internally attached springs, which may cause the resonator(s) to oscillate. Thus, the movement of the tremor may exert a force on the TMD system, and the movement of the resonators, which can destructively interfere with the oscillation of the hand tremors, may transmit the force to damp the tremors through this same mechanism.

Similarly, a crank or a crankshaft mechanism may be implemented to achieve this rotational-to-linear-motion, which also transmits the force from the tremors to the TMD device and vice versa. A mobile and/or computer application may be used in conjunction with this device for users to track parameters including but not limited to the amplitude, intensity, and/or frequency of their tremors over time. Accelerometers may be placed in one or more locations (for instance, on the resonators and/or on the distal part of the half-glove that covers the hand). After collecting the relevant data, the accelerometer may transfer this data to the on-board microcontroller (which may store this information, as may another external storage drive). This data may then be transferred to the mobile/computer application via a wireless module. The ability to track the amplitude, intensity, and/or frequency of tremors may provide users and/or physicians insight of the progression of the user's condition over time. It may also provide insight to physicians in case a change in medication and/or dosage is required for the user.

### Frictional Damping Mechanism

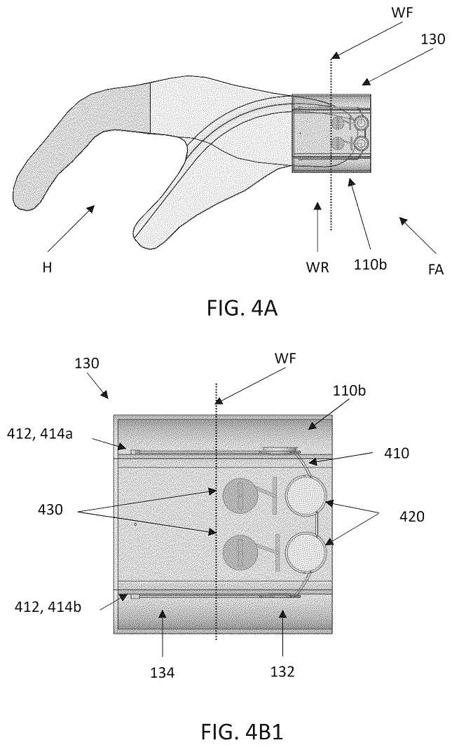

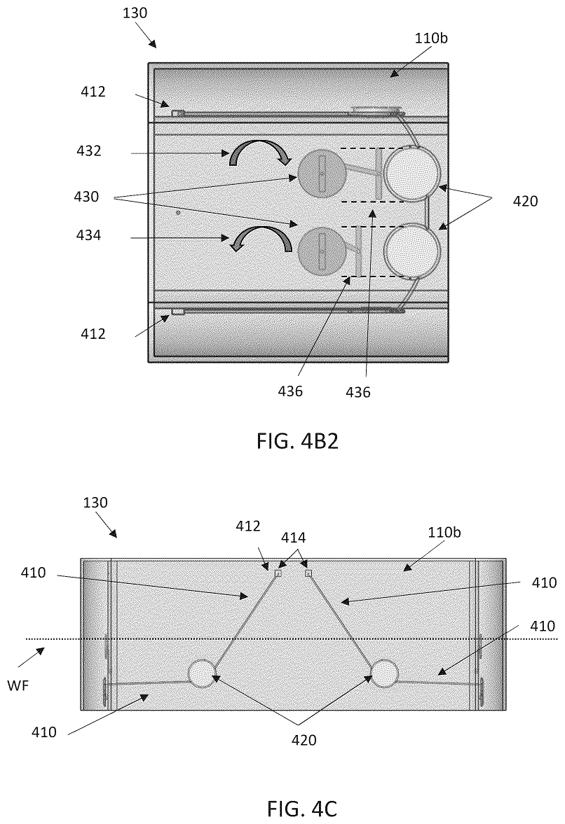

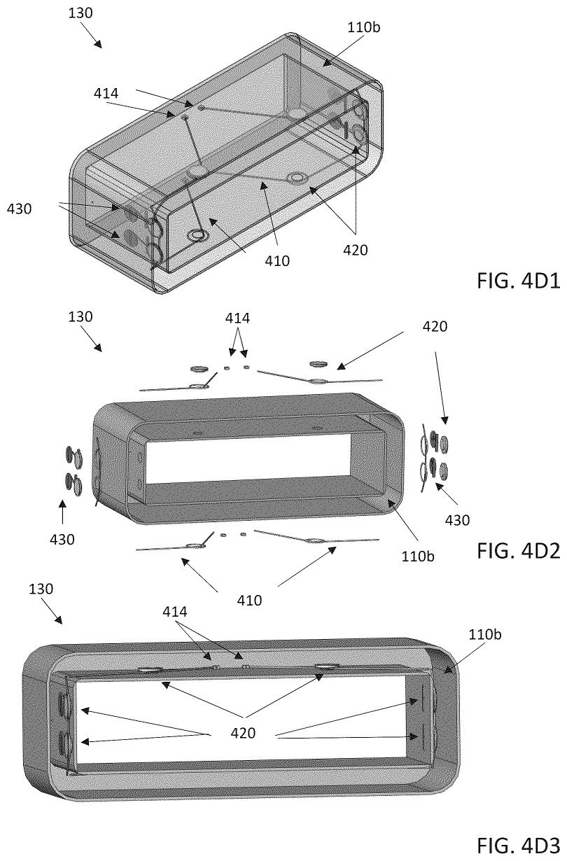

Referring now to FIGS. 4A-4D3, the frictional damping mechanism 130 is now described. The frictional damping mechanism 130 may be located inside the wearable half glove base 110, which covers parts of the hand HA, wrist WR, and distal forearm FA. As shown in FIG. 4A, the frictional damping mechanism 130 may be located at the proximal portion 110b of the wearable base.

The glove-like wearable base 110 may itself be made of a viscoelastic material (e.g., elastomers, Viton). A viscoelastic material is one that experiences both elastic and viscous behavior when, for instance, undergoing a deformation. As shown in FIG. 4A, the section of the wearable base to the right (or proximal) of the wrist-flexion axis WF may remain fixed in place when the tremors begin (henceforth referred to as the “fixed region” 132); the section to the left (or distal) of that axis WF may move with the tremors (henceforth named “moving region” 134). When the hand HA flexes upward during the tremor, the viscoelastic glove 110 may deform upwards as well. The return to its natural state, however, may follow a viscous, time-dependent strain. Thus, when the tremor flexes downwards and then again upwards, the viscoelastic material may still be recovering from the initial upward deformation. The viscoelastic recovery at this period may create an interference with the movement of the hand tremors and can contribute to its damping. A thicker glove will have a greater damping effect.

Materials suitable for the viscoelastic glove 110 may include: viscoelastic materials with high mechanical loss coefficients (tan delta) including but not limited to thermoset elastomers such as (poly) acrylic rubber, ethylene vinyl acetate rubber, fluoro elastomer (e.g. FEPM, FKM), perfluoro elastomer (e.g. FFKM), butyl/halobutyl rubber, nitrile rubber, natural rubber (15-42% carbon black), fluorosilicone, (FVMQ), and silicone (e.g. VMQ, heat cured, low hardness, 5-15% fumed silica); thermoplastics such as PVC (polyvinylchloride, flexible, plasticized, Shore A60/A65/A85), Ethylene Ethyl Acrylate copolymer (12-20% ethyl acrylate), Ethylene Vinyl Acetate (33% Vinyl Acetate), Ethylene methyl acrylate copolymer, and thermoplastic elastomers like polyvinyl chloride, elastomer (Shore A35/A75/A55); polymer foams such as polyurethane foam (e.g., polyester polyurethane elastomeric open cell foam), polyester polyurethane reticulated open cell filter foam, polypropylene structural foam, polypropylene closed cell foam; and, some synthetic polymers (e.g., nylon), to name a few examples. The viscoelastic material(s) can be wrapped around or coated/enclosed in one or more non-viscoelastic and/or other viscoelastic material(s). This wrapping may add a layer of thermal insulation and may prevent structural changes of the viscoelastic material within. A combination of one or more of these viscoelastic materials can also be used as the base material of the half-glove 110. In some embodiments, the viscoelastic material may be used in conjunction with flexoelectric materials (e.g., a layer of flexoelectric material may be placed underneath, inside, or above the viscoelastic layer). Flexoelectric materials are those which experience an electrical polarization due to an applied strain gradient. Flexoelectric polymers may be used due to their flexoelectric properties and flexibility. For instance, chloroprene rubber, polyamide, butyl rubber and PVC may be used. Thin layers of more rigid flexoelectric materials like ferroelectrics, dielectrics, and semiconductors (barium titanate, polystyrene, silicon to name a few) may also be incorporated. The deformation of the glove due to tremors can induce a strain gradient in the glove and therefore in the flexoelectric material as well. This strain gradient can cause electric polarization in the flexoelectric material. A displacement current may be harnessed, for instance, using electrodes that may be placed in or around the flexoelectric material/glove. One way in which this current can be used is in the converse flexoelectric effect: a voltage may be applied through an included capacitor, for instance, to induce a mechanical stress in the flexoelectric material opposite in direction to the mechanical stress caused by the tremors. The net effect may be to lower the amplitude of the tremors. This effect may take place when the tremors are occurring; a periodic deformation may be needed for this flexoelectric effect to occur, so this mechanism wouldn't restrict users' hand movements during normal tasks (as long as they don't cause periodic deformation of the flexoelectric material).

Inside the glove-like wearable base 110, a network of multiple capstans 420 and wires/belts/ropes (henceforth to as “wires” 410). The glove-like wearable base 110 will typically not be hollow; rather, the components depicted in FIGS. 4B1-4D3 may be embedded within the material of the wearable base 110. The capstans 420 may primarily be located in the fixed region 132—on the sides, top and bottom of the wearable base 110. Multiple wires 410 may be wrapped around the capstans 420; and, the wires 420 can be wrapped around each capstan 420 more than once. The capstans 420 can allow the wires 420 to move along them when the glove-like wearable base 110 is deformed by the tremors.

Fasteners 414 may be located in various locations at the moving region 134, for example, at the top and bottom of the glove-like wearable base 110, to hold the ends of the wires 410 fixed in place at various fixation points 412, as shown in FIGS. 4B1-4D3. As shown in FIG. 4D1, one end of the wire 410 may be located at the top of the glove-like wearable base 110 in the moving region 134. The wire 410 may then travel to the fixed region 132 where it can wrap around multiple capstans 420 along the way (for example, located in the top, sides, and bottom of the glove-like wearable base 110). After wrapping around and passing along the last capstan in the fixed region 132, the wire 420 can make its way back to the moving region 134, but at the bottom of the glove-like wearable base 110. There, the other end of the wire 420 can attach to the fastener(s) 414 at the bottom of the glove-like wearable base 110. The wires 420 are typically always in tension, whether the hand is tremoring or not.

Referring to FIG. 4B1, when hand tremors begin and the hand HA flexes upward, the distance from the top fastener 414a to the capstans 420 in the fixed region 132 may decrease, while the distance from bottom fastener 414b to the capstans 420 in the fixed region 132 may increase. This movement can cause the bottom fastener 414b to pull on the wire 410 and the wire 410 may slide along the capstans 420 clockwise. Similarly, the wire 410 may slide counterclockwise when the hand HA flexes downward. As the wire 410 slides along the capstan 420, the frictional force between the capstans 420 and the wire 410 wrapped around them can act in the direction opposite to that of the movement of the wire 410. This opposing force can act to damp the tremor force and therefore the amplitude of the tremor. A higher friction coefficient between the wire 410 and capstan(s) 420 can result in a higher frictional force. Moreover, the greater number of times the wire 410 wraps around the capstan(s) 420, the greater the length over which the frictional forces act, and the greater the damping effect.

Patients often have different tremor frequencies and amplitudes. Those with larger amplitude and/or higher frequency tremors, who would like further tremor reduction, can manually increase the effectiveness of the frictional damping mechanism 130. As shown in FIGS. 4B2, 4D1, and 4D2, multiple adjustment mechanisms 430, for example, slider-crank mechanisms, may be positioned in locations around the capstans 420. Users can rotate these adjustment mechanisms 430 using a connected knob. When the adjustment mechanism 430 is rotated clockwise as shown with directional arrow 432 in FIG. 4B2, a slider 436 can move linearly towards the capstan 420, and vice versa when rotated counterclockwise as shown with directional arrow 434 in FIG. 4B2.

The material of the glove-like wearable base 110 may be present between the adjustment mechanism(s) 430 and the capstan(s) 420. Thus, when the adjustment mechanism(s) 430 are rotated such that the slider(s) 436 moves towards the capstan(s) 436, the slider(s) 436 may first push and exert a force on the material of the wearable base 110. The material can in turn exert a force on the wire(s) 410 wrapped around the capstan(s) 420. This can increase the normal force acting on the wire(s) 410 and capstan(s) 420, which can result in a higher frictional force against that of the tremors. The more the crank is rotated clockwise, the greater the pressure on the material, the higher the normal force on the wire(s) 410 around the capstan(s) 420, and the higher frictional force to damp the tremors may be. Once the crank is rotated to the desired position, the user can push the knob inwards to hold the adjustment mechanism 430 in place. There may be small openings at different positions behind the crank that the knob fits into, depending on how much the crank is rotated. Pulling out the knob and rotating it counterclockwise can relieve the pressure on the material of the wearable base 110 and can decrease the resulting normal force on the wires/capstan systems. In this way, users can tune this adjustment mechanism(s) 430 to best suit their desired tremor reduction. In some embodiments, the additional pressure exerted on the glove material will also be partially felt in the user's hand. This feature can allow users to make the device 100 more effective when needed and revert it to a more comfortable position when there is less of a need to reduce their tremors. The frictional damping mechanism 130 will typically be effective in reducing tremors any time it is worn, even when the adjustment mechanism(s) 430 are at their lowest setting; users can simply calibrate the efficacy as they desire.

In some embodiments, there is no material between the adjustment mechanisms and the wires/capstans systems. In this case, the adjustment mechanism, when rotated one way, may exert a force directly on the wires/capstans systems. This rotation of the adjustment mechanism can likewise increase the normal force acting on the wires/capstans systems, which can result in a higher frictional force, and further damp the tremors. Similarly, pulling out the knob and rotating the knob counterclockwise can reduce the normal force on the wires/capstans systems, and thus lowering the frictional force and damping effect. In some embodiments, the additional pressure exerted on the wires/capstans systems can also be partially felt in the hand of the user.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus to treat tremor in an outer extremity of a subject, the apparatus comprising:
a wearable base configured to be worn over at least a joint of the outer extremity, the wearable base having a proximal fixed region and a distal moving region; and
a frictional damping mechanism coupled to the wearable base and configured to damp movement of the distal moving region relative to the proximal fixed region in response to tremor movement in the outer extremity,
wherein the frictional damping mechanism comprises at least one tension element held in tension within a body of the wearable base, and wherein, in response to the tremor movement, the at least one tension element applies a force opposite in direction to the tremor movement to damp the movement of the distal moving region relative to the proximal fixed region.

2. The apparatus of claim 1, wherein the outer extremity is a hand of the subject, and wherein the wearable base is configured to be worn over a wrist and the hand of the patient.

3. The apparatus of claim 1, wherein the frictional damping mechanism further comprises a viscoelastic material of the wearable base, the viscoelastic material being configured to deform and interfere with the tremor movement in response to the tremor movement.

4. The apparatus of claim 1, wherein the at least one tension element comprises at least one belt, wire, or rope.

5. The apparatus of claim 1, wherein the at least one tension element comprises a plurality of tension elements.

6. The apparatus of claim 1, wherein ends of the at least one tension element are fixedly attached to the distal moving region of the wearable base, and wherein the frictional damping mechanism further comprises at least one capstan at the proximal fixed region coupled to the at least one tension element.

7. The apparatus of claim 6, wherein the at least one tension element is wrapped around the at least one capstan.

8. The apparatus of claim 6, wherein the frictional damping mechanism further comprises at least one adjustment element coupled to the at least one capstan or fastener to increase or decrease an amount of tension the at least one tension element is held in within the wearable base.

9. The apparatus of claim 1, wherein the wearable base comprises a flexoelectric material.

10. The apparatus of claim 1, wherein the frictional damping mechanism further comprises at least one adjustment element coupled to the wearable base to increase or decrease the pressure the wearable base exerts on the at least one tension element.

11. The apparatus of claim 10, wherein the at least one adjustment element is controlled by one or more knobs proximate at least one capstan.

12. A method of treating tremor in an outer extremity of a subject, the method comprising:
providing a wearable base to be worn over at least a joint of the outer extremity; and
damping movement of a distal moving region of the wearable base worn on the outer extremity relative to a proximal fixed region of the wearable base in response to tremor movement in the outer extremity using a frictional damping mechanism,
wherein damping movement using the frictional damping mechanism comprises applying a force opposite in direction to the tremor movement in response to the tremor movement with the frictional damping mechanism.

13. The method of claim 12, wherein the wearable base is worn over a wrist and at least a portion of a hand of the subject.

14. The method of claim 12, wherein the force opposite in direction to the tremor movement is applied by a viscoelastic material of the wearable base, the viscoelastic material being configured to deform and interfere with the tremor movement in response to the tremor movement.

15. The method of claim 12, wherein the force opposite in direction to the tremor movement is applied by at least one tension element held in tension within a body of the wearable base.

16. The method of claim 15, further comprising adjusting an amount of tension of the at least one tension element.

17. The method of claim 16, further comprising at least one capstan at the proximal fixed region of the wearable base coupled to the at least one tension element, wherein adjusting an amount of tension of the at least one tension element comprises adjusting a number of times the tension element is wrapped around the at least one capstan.

18. The method of claim 12, further comprising measuring one or more characteristics of the tremor in the outer extremity of the subject.

19. The method of claim 12, further comprising applying voltage to induce mechanical stress in a flexoelectric material of the wearable base, wherein the mechanical stress is opposite in direction to the tremor movement.

20. The method of claim 12, further comprising adjusting a strength of frictional damping by rotating a knob connected to adjustment mechanisms disposed around at least one capstan at the proximal fixed region of the wearable base.
